SEQUENCE LISTING (12 sequences)

SEQ ID NO: 1 - DNA, 20 bp, Artificial sequence
  Single strand DNA primer
gaccaaggaagtgaagtggc

SEQ ID NO: 2 - DNA, 24 bp, Artificial sequence
  Single strand DNA primer
aggagaggtgaggctctggaaaac

SEQ ID NO: 3 - DNA, 23 bp, Artificial sequence
  Single strand DNA primer
cactatgggactgagtaacattc

SEQ ID NO: 4 - DNA, 23 bp, Artificial sequence
  Single strand DNA primer
gcactgacagttcagaattcatc

SEQ ID NO: 5 - DNA, 22 bp, Artificial sequence
  Single strand DNA primer
ctctgcagtgcgtcctctgggg

SEQ ID NO: 6 - DNA, 24 bp, Artificial sequence
  Single strand DNA primer
gatggtatcagaaacccctgtagc

SEQ ID NO: 7 - DNA, 24 bp, Artificial sequence
  Single strand DNA primer
tatcacccagatgattgggtcagc

SEQ ID NO: 8 - DNA, 24 bp, Artificial sequence
  Single strand DNA primer
ccagggttaccaagttgttgctca

SEQ ID NO: 9 - DNA, 22 bp, Artificial sequence
  Single strand DNA primer
atgaaggtctccgcggcagccc

SEQ ID NO: 10 - DNA, 22 bp, Artificial sequence
  Single strand DNA primer
ctagctcatctccaaagagttg

SEQ ID NO: 11 - DNA, 22 bp, Artificial sequence
  Single strand DNA primer
accatcaagctctgcgtgactg

SEQ ID NO: 12 - DNA, 23 bp, Artificial sequence
  Single strand DNA primer
gcaggtcagttcagttccaggtc

(19) United States
(12) Patent Application Publication
(10) Pub. No.: US 2003/0096016 A1
(43) Pub. Date: May 22, 2003

(54) Methods of kidney transplantation utilizing developing nephric tissue

(75) Inventors: Yair Reisner, Tel Aviv (IL); Benjamin Dekel, Tel-Aviv (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: YEDA RESEARCH AND DEVELOPMENT CO. LTD.

(21) Appl. No.: 10/118,933

(22) Filed: Apr. 10, 2002

Related U.S. Application Data
(60) Provisional application No. 60/317,452, filed on Sep. 7, 2001.

(51) Int. Cl. A61K 35/23

(52) U.S. Cl. 424/558; 435/29

(57) ABSTRACT

A method of treating a kidney disease in a subject is disclosed. The method is effected by transplanting into the subject a graft of nephric tissue at a predetermined developmental stage thereby treating the kidney disease in the subject.

### FIELD AND BACKGROUND OF THE INVENTION

The present invention relates to methods of treating kidney diseases. More particularly, the present invention relates to methods of treating kidney disease via transplantation of developing human or porcine nephric tissues.

Treatment of kidney disease via MHC haplotype-matched allogeneic kidney transplantation is a widely practiced, and often life-saving, therapeutic modality which, nevertheless, suffers from serious limitations.

The rarity of available donor organs and the necessity to obtain organs from histocompatible and morphologically compatible donors, which are often poorly represented in the donor pool, results in numerous renal failure related deaths each year.

In addition, even when histocompatible kidneys are available for transplantation, major immunosuppressive regimens are required in order to permit engraftment and tolerance of allogeneic organs. These regimens include mandatory administration of powerful immunosuppressant drugs, such as cyclosporine A, which cause severe side-effects such as carcinogenicity, nephrotoxicity and greatly weakening of the body's ability to fight infection.

Furthermore, kidney transplantation is limited in that patients having successfully undergone such procedures nevertheless sooner or later undergo acute graft rejection, thereby necessitating emergency surgical intervention to remove the graft followed by the necessity to be placed on kidney dialysis pending availability of another compatible organ for transplantation.

Finally, although cadaveric kidneys can be used, donor kidneys are often provided by graft recipient family members which must sacrifice one of their kidneys via a process of major surgery for organ removal.

Various approaches have been attempted or conceived in order to overcome these limitations.

One such approach envisages substituting transplantation of fully differentiated kidneys with transplantation of embryonic or fetal nephric cells or tissues. Such cells or tissues have been found to possess unique immunologic characteristics allowing prolonged survival in a non-syngeneic environment (Hammerman M R. (2000) Pediatr Nephrol. 14:513).

For example, it has been demonstrated that metanephric tissue from 12-22 week-old human fetuses, when transplanted into immunodeficient murine hosts, becomes vascularized, undergoes rapid growth and differentiates into functional nephrons (Dekel B. et al. (1997) Transplantation 64:1550; Dekel B. et al. (2001) J Am Soc Nephrol., in press)). When such experiments were modified by host-immune reconstitution with graft-allogeneic human PBMC, rejection of fetal kidney transplants was induced but, importantly, was shown to be delayed compared to that of adult kidney tissue allografts (Dekel B. et al. (1997) Transplantation 64:1550; Dekel B. et al. (1997) Transplantation 64:1541; Dekel B. et al. (1999) Int Immunol. 11:1673). Such metanephric grafts were shown to display reduced tissue apoptosis and destruction as well as a sustained growth phase (Dekel B. et al. (1997) Transplantation 64:550; Dekel B. et al. (2000) Transplantation 69:1470).

Thus, although the use of nephric tissue grafts from allogeneic embryos of fetuses was shown to be immunologically advantageous compared to that of adult kidneys from allogeneic donors for transplantation, graft rejection was merely delayed and not prevented. Another drawback of such an approach is that clinical application thereof may require prohibitively large amounts of human metanephric cells or tissues.

This drawback has been addressed in approaches for treatment of non-renal diseases via transplantation of xenogeneic porcine grafts. For example, transplantation of embryonic or fetal developing tissues has been attempted for treating Parkinson's disease, via transplantation of fetal ventral mesencephalic tissue (Subramanian, T. (2001) Semin Neurol. 21(1):103; Schumacher J M. et al., Neurology (2000) 54(5):1042) and pancreatic disease via transplantation of fetal islet cells (Otonkoski T. et al. (1999) Transplantation 68(11):1674; Groth C G. et al. (1999) J Mol Med. 77(1):153). Such studies, however, required administration of immunosuppressants to facilitate engraftment of transplanted tissues (Subramanian, T. (2001) Semin Neurol. 21(1):103; Schumacher J M. et al. (2000) Neurology 54(5):1042; Otonski et al. (1999) Transplantation 68(11):1674) or required pretransplant in vitro culture, under empirically-defined tissue-optimal conditions, of pancreatic tissue at an empirically-defined tissue-specific developmental stage. These empirically defined conditions, being specific to pancreatic tissue, a completely unrelated tissue type relative to nephric tissue, both functionally and anatomically, cannot be readily applied to optimal transplantation of developing nephric tissue without due experimentation. The latter study furthermore failed to demonstrate tolerance of transplanted grafts by human immune cells.

There is thus a widely recognized need for, and it would be highly advantageous to have, a method of treating kidney disease using nephric graft transplantation devoid of the above limitations.

### SUMMARY OF THE INVENTION

According to one aspect of the present invention there is provided a method of treating a kidney disease in a subject, the method comprising transplanting into the subject a graft of human nephric tissue being at a stage of differentiation corresponding to 4 to 10 weeks of gestation, thereby treating the kidney disease in the subject.

According to further features in preferred embodiments of the invention described below, the graft is selected not substantially expressing CD40, CD40L, or both CD40 and CD40L.

According to still further features in the described preferred embodiments, the selection is effected via RT-PCR analysis.

According to still further features in the described preferred embodiments, the graft is selected not substantially displaying expression of CD40, CD40L, or both CD40 and CD40L.

According to still further features in the described preferred embodiments, the selection is effected via RT-PCR analysis.

According to still further features in the described preferred embodiments, the graft is selected displaying less expression of at least one molecule than nephric tissue of human 14 week-old fetuses.

According to still further features in the described preferred embodiments, the at least one molecule is capable of stimulating or enhancing an immune response.

According to still further features in the described preferred embodiments, the at least one molecule is a lymphocyte coreceptor or a lymphocyte coreceptor ligand.

According to still further features in the described preferred embodiments, the at least one molecule is B7-1, CD40 or CD40L.

According to still further features in the described preferred embodiments, the subject is a human.

According to still further features in the described preferred embodiments, the graft of human nephric tissue is transplanted into the renal capsule, the kidney, the testicular fat, the sub-cutis, the omentum or the intra-abdominal space of the subject.

According to still further features in the described preferred embodiments, the method of treating a kidney disease in a subject further comprises treating the subject with an immunosuppressive regimen, thereby promoting engraftment of the graft of human nephric tissue in the subject.

According to still further features in the described preferred embodiments, treating the subject with an immunosuppressive regimen is effected prior to, concomitantly with or following the transplanting into the subject the graft of human nephric tissue.

According to still further features in the described preferred embodiments, treating the subject with an immunosuppressive regimen is effected by administration of an immunosuppressant drug and/or administration of an immune tolerance-inducing cell population.

According to still further features in the described preferred embodiments, the stage of differentiation corresponds to 5 to 9 weeks of gestation.

According to still further features in the described preferred embodiments, the stage of differentiation corresponds to 6 to 9 weeks of gestation.

According to still further features in the described preferred embodiments, the stage of differentiation corresponds to 7 to 8 weeks of gestation.

According to still further features in the described preferred embodiments, the stage of differentiation corresponds to 7 weeks of gestation.

According to still further features in the described preferred embodiments, the stage of differentiation corresponds to 8 weeks of gestation.

According to another aspect of the present invention there is provided a method of treating a kidney disease in a subject, the method comprising transplanting into the subject a graft of porcine nephric tissue being at a stage of differentiation corresponding to 3 to 6 weeks of gestation, thereby treating the kidney disease in the subject.

According to further features in preferred embodiments of the invention described below, the graft is selected not substantially displaying expression of CD40, CD40L or both CD40 and CD40L.

According to still further features in preferred embodiments of the invention described below, the selection is effected via RT-PCR analysis.

According to still further features in preferred embodiments of the invention described below, the graft is selected displaying less expression of at least one molecule than nephric tissue of porcine fetuses at a developmental stage equivalent to that of nephric tissue of human 14 week-old fetuses.

According to still further features in preferred embodiments of the invention described below, the selection is effected via RT-PCR analysis.

According to still further features in preferred embodiments of the invention described below, the at least one molecule is capable of stimulating or enhancing an immune response.

According to still further features in preferred embodiments of the invention described below, the at least one molecule is a lymphocyte coreceptor or a ligand of a lymphocyte coreceptor.

According to still further features in preferred embodiments of the invention described below, the at least one molecule is B7-1, CD40 or CD40L.

According to still further features in preferred embodiments of the invention described below, the graft of porcine nephric tissue is transplanted into the renal capsule, the kidney, the testicular fat, the sub-cutis, the omentum or the intra-abdominal space of the subject.

According to still further features in the described preferred embodiments, the method of treating a kidney disease in a subject further comprises treating the subject with an immunosuppressive regimen, thereby promoting engraftment of the graft of porcine nephric tissue in the subject.

According to still further features in the described preferred embodiments, treating the subject with an immunosuppressive regimen is effected prior to, concomitantly with or following the transplanting into the subject the graft of porcine nephric tissue.

According to still further features in the described preferred embodiments, the stage of differentiation corresponds to 4 to 5 weeks of gestation.

According to still further features in the described preferred embodiments, the stage of differentiation corresponds to 4 weeks of gestation.

According to still further features in the described preferred embodiments, the stage of differentiation corresponds to 5 weeks of gestation.

The present invention successfully addresses the shortcomings of the presently known configurations by providing a method of successfully transplanting nephric tissues without adjunct immunosuppressive treatment.

According to yet another aspect of the present invention there is provided a method of evaluating the transplantation suitability of a tissue explant or cell culture comprising testing cells of the tissue explant or cells of the cell culture for expression of at least one molecule, thereby evaluating the transplantation suitability of the tissue explant or cell culture.

According to further features in preferred embodiments of the invention described below, the testing is effected via RT-PCR analysis.

According to further features in preferred embodiments of the invention described below, the at least one molecule is capable of stimulating or enhancing an immune response.

According to further features in preferred embodiments of the invention described below, the at least one molecule is a lymphocyte coreceptor or a ligand of a lymphocyte coreceptor.

According to still further features in the described preferred embodiments, the at least one molecule is CD40, CD40L or B7-1.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-d are data plots depicting the effect of alloreactive human PBMC on growth of transplanted nephric tissues from 7-, 8-, 10- and 14 week human embryos or fetuses (FIGS. 1a-d, respectively). Growth was measured 4, 6 and 8 weeks following transplantation of immune cell reconstituted and non-immune cell reconstituted (triangles and squares, respectively) animals.

FIGS. 2a-h are photographs depicting the differential effect of alloreactive human PBMC on human nephric tissue transplants. FIGS. 2a-d depict the deleterious effects of PBMC on transplants originating from 14-week fetuses. FIG. 2a is a macroscopic view of the transplant (arrow) 8 weeks following transplantation. FIG. 2b is a photomicrograph depicting immunostaining of human CD3 in transplanted nephric tissue (10× original magnification). FIGS. 2c-d are photomicrographs depicting immunostaining of human CD3 in a glomerulus and a renal tubule, respectively, of transplanted nephric tissue (40× original magnification). FIGS. 2e-g depict fully tolerated transplants originating from 8-week embryos. FIG. 2e is a representative macroscopic view of a transplanted metanephros (arrow) 8 weeks following transplantation. FIG. 2f is a photomicrograph depicting hematoxylin and eosin (H+E) histological staining of transplanted nephric tissue (10× original magnification). FIGS. 2g-h are photomicrographs depicting immunostaining of human CD3 in a glomerulus and a renal tubule, respectively, of transplanted nephric tissue (40× original magnification).

FIG. 3 is a photostereomicrograph depicting a large urine-like fluid-filled cyst formed by intra-abdominal transplants of human nephric tissue transplanted in mice reconstituted with graft-allogeneic human PBMC.

FIGS. 4a-c depict analysis of mRNA expression of co-stimulatory molecules in normal human developing kidneys, in developing human kidneys immediately following transplantation, but prior to administration of allogeneic human PBMC, and at 2, 4, and 6 weeks following reconstitution of graft recipients with graft-allogeneic human PBMC. FIGS. 4a-c depict nephric tissue transplants originating from 8-, 14- and 22-week fetuses, respectively.

FIGS. 5a-d are data plots depicting the effect of human PBMC on growth of transplanted nephric tissue from 3, 4, 6 and 8 week-old (FIGS. 5a-d, respectively) porcine embryos or fetuses. Measurements were made 4, 6 and 8 weeks posttransplant of PBMC- and non-PBMC-reconstituted animals (triangles and squares, respectively).

FIGS. 6a-c are photomicrographs depicting rejection of transplanted adult porcine kidney tissue by human PBMC. FIGS. 6a-b are ×4 and ×20 magnification views, respectively, depicting hematoxylin and eosin (H+E) histological staining of subcapsular adult porcine kidney transplants 4 weeks following intraperitoneal infusion of human PBMC. FIG. 6c depicts immunostaining of human CD3 in transplanted tissue.

FIGS. 7a-h are photographs depicting the differential effect of xenoreactive human PBMC on porcine nephric tissue transplants. FIGS. 7a-d depict the deleterious effects of PBMC on transplants originating from 8-week fetuses. FIG. 7a is a macroscopic view of the transplant (arrow) 8 weeks following transplantation. FIG. 7b is a photomicrograph depicting H+E histological staining of transplanted nephric tissue (lox original magnification). FIGS. 7c-d are photomicrographs depicting immunostaining of human CD3 in damaged blood vessels and tubules of transplanted tissue (40× original magnification). FIGS. 7e-g depict fully tolerated transplants originating from 4-week embryos. FIG. 7e is a macroscopic view of transplanted nephric tissue (arrow) 8 weeks posttransplant. FIG. 7f is a photomicrograph depicting H+E histological staining of transplanted nephric tissue (10× original magnification). FIGS. 7g-h are photomicrographs depicting immunostaining of human CD3 in glomeruli and tubuli, (respectively), of transplanted nephric tissue (40× original magnification). These structures are intact and do not contain infiltrating human CD3 cells.

FIGS. 8a-c are photomicrographs depicting pluripotency of porcine nephric tissue from 3 week-old embryos 8 weeks following subcapsular transplantation in conjunction with transplantation of human PBMC. FIG. 8a depicts H+E histological analysis of tissue at low magnification (4× original magnification) showing blood vessels (upper right arrow), cartilage (upper left arrow) and bone (lower arrow). FIGS. 8b-c depict H+E stained tissue at high magnification (40× original magnification) showing bone and cartilage, respectively.

FIG. 9 is a photostereomicrograph depicting a large urine-like fluid-filled cyst formed by an intra-abdominal transplant of porcine nephric tissue transplanted in recipients reconstituted with xenoreactive human PBMC.

### DESCRIPTION OF THE PREFERRED EMBODIMENTS

The present invention is of methods of treating kidney diseases and methods of evaluating the transplantation suitability of grafts. Specifically, the present invention relates to allogeneic human or porcine developing nephric tissue grafts for use in transplantation, which grafts, selected displaying low immune coreceptor expression levels, grow and differentiate into functional nephric organs which are fully tolerated by human alloreactive or xenoreactive effectors, respectively. As such, when transplanted into a recipient, the immune system of which containing such alloreactive or xenoreactive human effectors, such developing nephric tissue grafts form functional nephric organs.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

Developing allogeneic human or porcine nephric tissue grafts capable of growth and development in a recipient, the immune system of which containing alloreactive of xenoreactive human effectors, respectively, have been described by the prior art.

For example, nephric tissue grafts derived from 12- to 22-week-old human fetuses transplanted into immunodeficient murine hosts bearing graft-alloreactive human effectors, have been shown to become vascularized and to undergo growth and differentiation into functional nephric organs displaying delayed, but not prevented, graft rejection as compared to adult-stage kidney grafts.

In another approach, porcine fetal islet cell grafts transplanted into immunodeficient mice were shown to differentiate and mature following transplantation, however the grafts in these studies were not demonstrated to be tolerated by human graft-immunoreactive effectors. Furthermore optimal in vivo differentiation of such grafts required pre-transplant in vitro culture under pancreatic tissue-specific conditions empirically defined following extensive experimentation. Yet further extensive experimentation was required in order to determine the optimal pancreatic tissue-specific developmental stage of the fetus from which grafts must be derived for optimal engraftment and differentiation.

All other approaches employing porcine fetal tissues in the presence of human graft-immunoreactive effectors required adjunct immunosuppression by administration of highly toxic, cellular immunity-impairing immunosuppressive drugs to prevent graft rejection.

Thus, all prior art approaches employing transplantation of developing nephric tissue grafts have failed to provide adequate solutions for development thereof into fully tolerated, functional nephric organs following transplantation into a recipient having an immune system containing non-graft syngeneic human graft-reactive immune effectors.

While reducing the present invention to practice, the present inventors have identified a unique developmental stage of embryos or fetuses from which nephric tissue grafts capable of growing and differentiating into fully tolerated, functional, nephric organs following transplantation into a subject can be derived.

Utilizing such nephric tissues, the method of the present invention can be employed to treat a kidney disease in a subject without having to employ immunosuppressive treatment.

It will be understood by one well versed in the art, that non-syngeneic grafts can be derived from both an allogeneic source, being of the same species as the recipient, as well as from a xenogeneic source, being of a different species as the recipient.

According to the method of the present invention, a fully tolerated graft is a graft which is not rejected or rendered non-functional by cells of the host's immune system, such as neutrophils or T lymphocytes.

As described in the Examples section below, nephric graft functionality is characterized by production of fluid containing supra-plasma concentrations of urine-specific byproducts, such as, for example, urea nitrogen and creatinine.

Thus, according to one aspect of the present invention there is provided a method of treating a kidney disease in a mammal, preferably a human.

Examples of kidney diseases which can be treated by the present invention include, but are not limited to, acute kidney failure, acute nephritic syndrome, analgesic nephropathy, atheroembolic renal disease, chronic kidney failure, chronic nephritis, congenital nephrotic syndrome, end-stage renal disease, Goodpasture's syndrome, IgM mesangial proliferative glomerulonephritis, interstitial nephritis, kidney cancer, kidney damage, kidney infection, kidney injury, kidney stones, lupus nephritis, membranoproliferative glomerulonephritis I, membranoproliferative glomerulonephritis II, membranous nephropathy, necrotizing glomerulonephritis, nephroblastoma, nephrocalcinosis, nephrogenic diabetes insipidus, IgA-mediated nephropathy, nephrosis, nephrotic syndrome, polycystic kidney disease, post-streptococcal glomerulonephritis, reflux nephropathy, renal artery embolism, renal artery stenosis, renal papillary necrosis, renal tubular acidosis type I, renal tubular acidosis type II, renal underperfusion and renal vein thrombosis.

The method of treating a kidney disease of the present invention is effected by transplanting into a subject a graft of human or porcine developing nephric tissue.

The anatomical location of such transplants varies with the nature and severity of the disease treated. The nephric tissue can be transplanted into the renal capsule, the kidney or the intra-abdominal space of the subject.

For example, subcapsular transplants enable insertion of a catheter requiring only a short extension to the skin where urine can be collected and with intra-abdominal transplants, the developing ureter or the renal pelvis of the nephric tissue transplant can be anastomosed to the host's excretory system. Alternately, grafts can be transplanted in the testicular fat, the sub-cutis or the omentum, according to need.

According to one preferred embodiment of the present invention, as described in the Examples section below, the graft is transplanted into the renal capsule of the subject.

According to another preferred embodiment of the present invention, as described in the Examples section, below, the graft is transplanted into the intra-abdominal space of the subject.

The nephric tissue utilized by the present invention is preferably derived from an embryo or a fetus, although the use of nephric tissue which is derived in vitro from cultured precursor cells, such as, but not limited to, embryonic stem cells or embryonic nephric progenitor cells is also contemplated by the present invention.

When utilizing human nephric tissue, such tissue is preferably at a stage of differentiation corresponding to 4 to 10, preferably 5 to 9, more preferably 6 to 9 or most preferably 7 to 8 weeks of gestation.

As demonstrated by the results presented in the Examples section below, successful transplantation was achieved using human developing nephric tissue at a stage of differentiation corresponding to 8 weeks of gestation.

Alternately, treatment of kidney disease, according to another aspect of the present invention is effected by transplanting into the subject a graft of porcine developing nephric tissue being at a stage of differentiation corresponding to 3 to 6 or preferably 4 to 5 weeks of gestation.

Most preferably, as shown in the Examples section below, porcine developing nephric tissue is transplanted at a stage of differentiation corresponding to 4 weeks of gestation.

It will be understood by one versed in the art that a period of gestation corresponds to a time-period elapsed since fertilization of a developing embryo or fetus. Thus, the stage of differentiation of a developing nephric tissue graft corresponds to the developmental stage of the embryo or fetus from which it is derived. In the case of in vitro culture derived developing nephric tissue, the stage of differentiation thereof corresponds to that of the embryo or fetus from which nephric tissue at a similar stage of development can be derived.

It will also be understood by one versed in the art that developing nephric tissue can include whole developing kidneys or parts thereof, including individual cells, pronephric, mesonephric or metanephric tissue as well as any tissue type which is committed to develop along a nephric tissue lineage.

In order to minimize rejection of human grafts, transplantation is preferably effected using grafts selected displaying less expression of at least one molecule capable of stimulating or enhancing immune responses than nephric tissue of human 14 week-old fetuses.

In the case of porcine grafts, in order to minimize graft rejection transplantation is preferably effected using grafts selected displaying less expression of at least one molecule capable of stimulating or enhancing immune responses than nephric tissue of porcine fetuses at a developmental stage equivalent to that of nephric tissue of human 14 week-old fetuses.

As used herein, “expression” of a molecule is defined as the presence of mRNA and/or protein of such a molecule in cellular and/or extracellular biological materials, such as grafts.

According to a preferred embodiment, selecting of grafts is effected by analyzing expression of mRNA species in grafts.

Preferably, analysis of mRNA in biological materials such as grafts is performed by RT-PCR analysis, as described in the Materials and Methods section of Example 1 of the Examples section, below.

Alternately, analysis of mRNA expression can be performed using any method of mRNA analysis having equivalent detection sensitivity as the aforementioned RT-PCR method, such as, for example, RT-PCR using different protocols than the one described in Example 1 of the Examples section, below, Northern blotting or microarray chip hybridization.

Methods of detecting the presence of proteins in materials such as grafts are well known to those of ordinary skill in the art and include, for example, Western immunoblotting analysis, fluorescent flow cytometry (FACS), fluorescent in situ hybridization (FISH), ELISA, microarray chip hybridization, and the like.

Examples of molecules capable of stimulating or enhancing immune responses include cytokines, chemokines, inflammatory mediators, and immune cell receptors or soluble or membranal ligands of immune cell receptors.

Examples of immune cells include B lymphocytes, T lymphocytes, dendritic cells, antigen presenting cells (APCs), macrophages, monocytes, granulocytes, mast cells, neutrophils and the like.

According to a preferred embodiment, human grafts are selected displaying less expression of at least one lymphocyte coreceptor, or ligand thereof, than nephric tissue of human 14 week-old fetuses and porcine grafts are selected displaying less expression of at least one lymphocyte coreceptor, or ligand thereof, than nephric tissue of porcine fetuses at a developmental stage equivalent to that of nephric tissue of human 14 week-old fetuses.

Examples of lymphocyte coreceptors and their ligands include CD28 and B7-1 (CD80) or B7-2 (CD86), CD40 and CD40L (CD40 ligand, CD154), CD2 and CD58 (lymphocyte function associated antigen-3, LFA-3), and ICAM-1 (intercellular adhesion molecule-1) and LFA-1 (lymphocyte function associated antigen-1).

According to one preferred embodiment, grafts are selected not substantially displaying expression of CD40 or CD40L, preferably CD40 and CD40L.

According to another preferred embodiment, grafts are selected displaying less expression of B7-1 than nephric tissue of human 14 week-old fetuses and porcine grafts are selected displaying less expression of B7-1 than nephric tissue of porcine fetuses at a developmental stage equivalent to that of nephric tissue of human 14 week-old fetuses.

According to a most preferred embodiment, human grafts are selected not substantially displaying expression of CD40 and CD40L, and displaying less expression of B7-1 than nephric tissue of human 14 week-old fetuses; and porcine grafts are selected not substantially displaying expression of CD40 and CD40L, and displaying less expression of B7-1 than nephric tissue of porcine fetuses at a developmental stage equivalent to that of nephric tissue of human 14 week-old fetuses.

Example 1, of the Examples section which follows, shows that human grafts not substantially displaying expression of CD40 and CD40L and expressing less B7-1 than nephric tissue of 14-week human fetuses are not rejected by the host. As such, these grafts are most suitable for use as graft tissue.

Although the results shown in the Examples section (below) indicate that the method of the present invention can be employed to perform successful transplantation without any form of immunosuppression whatsoever, there may arise instances in which adjunct immunosuppression is medically indicated. In such cases, the method of the present invention, having been shown to be superior to prior art methods with respect to avoidance of graft rejection, affords the use of minimal adjunct immunosuppression, such as administration of highly toxic immunosuppressive agents. The method of the present invention therefore affords the use of adjunct immunosuppressive treatment producing fewer, if any, side-effects relative to adjunct immunosuppressive treatment associated with prior art transplantation methods.

Thus, the method of treating a subject suffering from kidney disease, according to the present invention, may comprise an additional step of treating the subject, prior to, during or following transplantation, with an immunosuppressive regimen such as administration of an immunosuppressive agent and/or a graft donor-derived tolerance-inducing cell population.

Examples of immunosuppressive agents include, but are not limited to, CTLA4-Ig, anti-CD40 antibodies, anti-CD40 ligand antibodies, anti-B7 antibodies, rapamycin, prednisone, methyl prednisolone, azathioprine, cyclosporine A, cyclophosphamide and fludarabin.

Examples of tolerance-inducing cell populations include, but are not limited to, cells displaying a myeloid phenotype, cells displaying the surface marker CD33, veto cells and CD8⁺ T cells.

It is expected that during the life of this patent many relevant medical diagnostic techniques will be developed and the scope of the term analytic mechanism is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8^{th} Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Transplants of Developing Nephric Tissue Obtained from 7-8 Week Human Fetuses Develop into Functional Nephric Organs Which are Fully Tolerated by Alloreactive Human PBMC

Minimization, or preferably complete avoidance, of human kidney allograft rejection constitutes a highly desired therapeutic goal for treatment of kidney disorders. Prior art approaches have shown that developing nephric tissue allografts induce attenuated alloimmune responses in comparison to adult-stage kidney allografts. While conceiving the present invention, it was hypothesized that the earliest developmental stage during which developing nephric tissue is sufficiently differentiated to develop into functional nephric organs following transplantation corresponds to the developmental stage during which alloimmune rejection of such grafts is optimally minimized or, possibly, completely eliminated. Thus, while reducing the present invention to practice, experiments identifying such an optimal stage of development in human developing nephric tissue were performed by examining the fate of developing nephric tissue originating from various stages of gestation following transplantation into immunodeficient mice reconstituted with allogeneic human PBMC, as described below.

Materials and Methods:

Preparation of murine transplant hosts: Three month old Balb/c mice (Harlan Olac, Shaw's Farm, Blackthorn, Bicester, Oxon., UK) were used as hosts for the transplantation studies. All mice were kept in small cages (5-10 animals in each cage) and fed sterile food and acid water containing ciprofloxacin (20 mg/ml). Mice were exposed to split-dose total body irradiation (TBI; 3.5 Gy followed 3 days later by 9.5 Gy) by a 150-A ⁶⁰Co γ-beam source (produced by the Atomic Energy Commission of Canada, Kanata, Ontario) with a focal skin distance of 75 cm and a dose rate of 0.7 Gy/minute, as previously described (I. Lubin et al. (1994) Blood 83:2368). Bone marrow cells from NOD/SCID (severe combined immunodeficiency) mice (Weizmann Institute Animal Breeding Center, Rehovot, Israel) were flushed from femur and tibia shafts of 4-8 week-old mice, as previously described (M. Levite et al. (1995) Cell Immunol. 162:138). Recipient Balb/c mice were reconstituted with 3×10 SCID bone marrow cells administered intravenously in 1 ml PBS one day following the second fraction of TBI. The resulting SCID-like animals allowed excellent engraftment of functioning human hematopoietic cells or solid tissues (H. Marcus et al. (1995) Blood 86:398; H. Segall et al. (1996) Blood 88:88; Y. Reisner, S. Dagan (1998) Trends Biotechnol. 16:242; W. 0. Bocher, et al. (2001) Eur J Immunol. 31:2071).

Harvesting of developing nephric tissue: Developing human nephric tissues were obtained by curettage with the approval of a Helsinki committee and metanephroi were surgically dissected from embryos under a dissecting microscope as previously described (Rogers S. et al. (1998) Kidney Int. 54:27).

Transplantation of developing nephric tissue: Developing human nephric tissues were transplanted 7-10 days following reconstitution of irradiated hosts with SCID bone marrow, as follows. Nephric tissues were maintained in sterile conditions at 4° C. for approximately two hours in either RPMI 1640 or Dulbecco's Modified Eagle Medium supplemented with 10% fetal calf serum (Biological Industries, Beit Haemek, Israel). Transplantation of nephric tissues was performed under general anaesthesia induced by intraperitoneal injection of 2.5% Avertin in PBS (10 ml per kg body weight). Both host kidneys were exposed via a bilateral incision, a 1.5 mm incision was made at the caudal end of the kidney capsule and a 1 mm³ fragment of nephric tissue was implanted under each kidney capsule. Nephric tissues were also transplanted intra-abdominally to control for the possibility that immune privilege is renal sub-capsular space-specific. In some experiments, nephric tissues were implanted and sutured (5-0 suture) onto the testicular fat pad. Transplanted mice were treated post-operatively with ciprofloxacin in their drinking water for 7 days.

Engraftment of mice with human PBMC: One to three days following 8 transplantation of nephric tissue, as described above, 10 human PBMC were injected intraperitoneally in host mice. Human PBMC were generated from Buffy coats obtained from normal volunteers as follows. Blood samples were overlayed on a cushion of Lymphoprep solution (Nycomed, Oslo, Norway) and centrifuged at 2000 rpm for 20 min, the interface layer was collected and washed twice, and cells were counted and resuspended in PBS (pH 7.4) at the desired concentration. Control mice did not receive human PBMC. For analysis of human lymphocyte engraftment, cells were recovered from peritonea 10-14 days following PBMC infusion. Single-cell suspensions were incubated for 30 min on ice with labelled anti-human CD3-PE and CD45-PerCP (pan-human leukocyte antigen) antibodies (Becton-Dickinson, Mountain View, Calif.). After washing, two- or three-color fluorescent analysis of human antigens was performed using a FACScan analyzer (Becton-Dickinson). Data was collected from lymphocytes selectively gated via standard forward- and side-scatter characteristics.

Analysis of graft infiltration, growth and differentiation: Human immune cell infiltration as well as growth and development of the developing nephric tissue into mature glomeruli and tubuli were monitored following transplantation, as follows.

Graft recipients were sacrificed 4, 6, 8 and 10 weeks following administration of human PBMC. Nephric tissue implants were initially assessed for engraftment and growth by macroscopic examination (color, diameter) of the transplants at the subcapsular or intra-abdominal site. Kidneys and their capsules were then removed and fixed in 10% paraffin.

For visualization of nephric structures, grafts were sectioned and mounted on slides coated with poly-L-lysine and sections were H+E stained for evaluation of graft differentiation, cellular infiltration, and tissue damage. Assessment of graft development was then performed by counting the number of mature glomeruli and tubuli in 10 consecutive high-power fields (HPF; 40× magnification) per transplant in 3 transplants per group. Transplant growth was assessed by determining the posttransplant:pretransplant size ratio for at least 3 transplants per group at each time point.

Human T cell infiltration in the grafts was quantitated by staining of CD3⁺ cells in sections, as previously described (M. T. Naveh et al. (1992) J Clin Invest. 90:2434), and counting the number of CD3⁺ cells in 10 consecutive microscope fields (100× magnification) per transplant in 3 transplants per group. In this case, paraffin tissue blocks of transplants were cut 4-6 μm thick, deparaffinized in xylene, rehydrated and placed for 15 min in ethanol containing 3% H₂O₂ to block endogenous peroxidase. Slides were thoroughly washed with tap water and transferred to PBS. Sections were then treated with 1% bovine serum albumin to prevent background staining and incubated for 1 h with rabbit anti-human CD3 antibody (pan T-cell; Dako) at room temperature in a humidified chamber. Slides were rinsed with PBS for 3 min and incubated with a biotinylated anti-rabbit antibody for 30 min and then incubated with peroxidase-conjugated streptavidin for 30 min (StrAvigen; Biogenex, San Ramon, Calif.). After rinsing, the peroxidase label was visualized by incubation with for 15 min and counterstained with Mayer's hematoxylin using an immunohistochemical staining kit according to the manufacturer's instructions (Biomeda, Foster City, Calif.). The reagent 3-amino-9-ethylcarbazol produced a red product that is soluble in alcohol and can be used with an aqueous mounting medium (Kaiser's glycerol gelatin). A negative control for staining of T lymphocytes was performed by following all of the aforementioned steps but omitting addition of primary antibody. Staining was found to be uniformly negative in transplants from control mice not infused with human PBMC.

Analysis of costimulatory molecule expression in grafts: Among the multiple co-stimulatory pathways identified, increasing evidence suggests that interaction of the T cell costimulatory receptors CD28 and CD40 ligand (CD40L, CD154) with their respective ligands B7-1/B7-2 and CD40 expressed on antigen presenting cells (APCs) are critical for T cell responses to alloantigens (M. H. Sayegh, L. A. Turka (1998) N Engl J Med. 338:1813).

Experiments were thus performed to test whether human immune cells did not reject grafts of allogeneic human nephric tissue from 8-week fetuses as a result of such nephric tissues downregulating expression of co-stimulatory molecules, as follows.

Expression of B7-1, B7-2, CD40 and CD40L mRNA in grafted nephric tissues originating from 8, 14, and 22-week fetuses was analyzed via RT-PCR prior to transplantation, immediately following transplantation but prior to allogeneic human PBMC infusion, and at 2, 4, and 6 weeks following reconstitution of mice with human PBMC.

Nephric tissues were homogenized with a glass-Teflon tissue homogenizer in Tri-reagent (Molecular Research Center, INC, Cincinnati, Ohio) for isolation of total RNA, according to the manufacturer's instructions. The isolated total RNA was air-dried, resuspended in nuclease-free water and quantified by spectrophotometry. Aliquots of 1 μg of total RNA were reverse-transcribed into cDNA using AMV reverse transcriptase according to standard procedures. Reverse transcription reaction cDNA product was diluted 1:50, 1:100, and 1:500 in sterile water and PCR amplification of costimulatory receptor sequences was performed using thermostable Tfl DNA polymerase in a 50 μl reaction mixture containing 40 μM of each dNTP, 0.4 μM of each primer, 10 mM Tris HCl (pH 8.3) and 1.5 mM MgCl₂. In all experiments the possibility of amplification from contaminating DNA was eliminated via control reactions in which reverse transcriptase was omitted from, or buffer alone was added to, the reverse transcription reaction mixture. Homology searches for all primer sequences were performed using the National Center for Biotechnology Information (NCBI) GenBank library to ensure primer specificity for the relevant human but not the corresponding mouse genes. Furthermore, in order to minimize non-specific amplification of non-target sequences, the PCR annealing temperature was set high (64° C.). The following sense and antisense primers, respectively in order presented, were used for PCR amplification: B7-1, 5′-GACCAAGGAAGTGAAGTGGC-3′ (SEQ ID NO:1) and 5′-AGGAGAGGTGAGGCTCTGGAAAAC 3′ (SEQ ID NO:2); B7-2, 5′-CACTATGGGACTGAGTAACATTC-3′ (SEQ ID NO:3) and 5′-GCACTGACAGTTCAGAATTCATC-3′ (SEQ ID NO:4); CD40, 5′-CTCTGCAGTGCGTCCTCTGGGG-3′ (SEQ ID NO:5) and 5′-GATGGTATCAGAAACCCCTGTAGC-3′ (SEQ ID NO:6); CD40L, 5′-TATCACCCAGATGATTGGGTCAGC-3′ (SEQ ID NO:7) and 5′-CCAGGGTTACCAAGTTGTTGCTCA-3′ (SEQ ID NO:8); HLA-DR, 5′-ATGAAGGTCTCCGCGGCAGCCC-3′ (SEQ ID NO:9) and 5′-CTAGCTCATCTCCAAAGAGTTG-3′ (SEQ ID NO:10); β-actin, 5′-ACCATCAAGCTCTGCGTGACTG-3′ (SEQ ID NO:11) and 5′-GCAGGTCAGTTCAGTTCCAGGTC-3′ (SEQ ID NO:12). In order to detect PCR signals in the linear phase of product amplification, 20-35 thermal cycles were performed per PCR reaction. Products of PCR reactions were separated electrophoretically in 1.5% agarose gel, stained with ethidium bromide and photographed under UV illumination, as previously described (V. K. Sharma et al. (1996) Transplantation 62:1860). Transcription of costimulatory molecules in tissue samples to be compared were amplified in parallel using a single master reagent mix. Each sample was tested at least three times.

Experimental Results:

During preliminary experiments to establish baseline experimental conditions, infusion of 10⁸ human PBMC was determined to be the minimal number capable of inducing complete rejection of human adult kidney tissue transplants engrafted into recipient mice (data not shown). Four weeks following such infusion, transplants were found to be massively infiltrated and graft tissue destruction and rejection were apparent (Table 1).

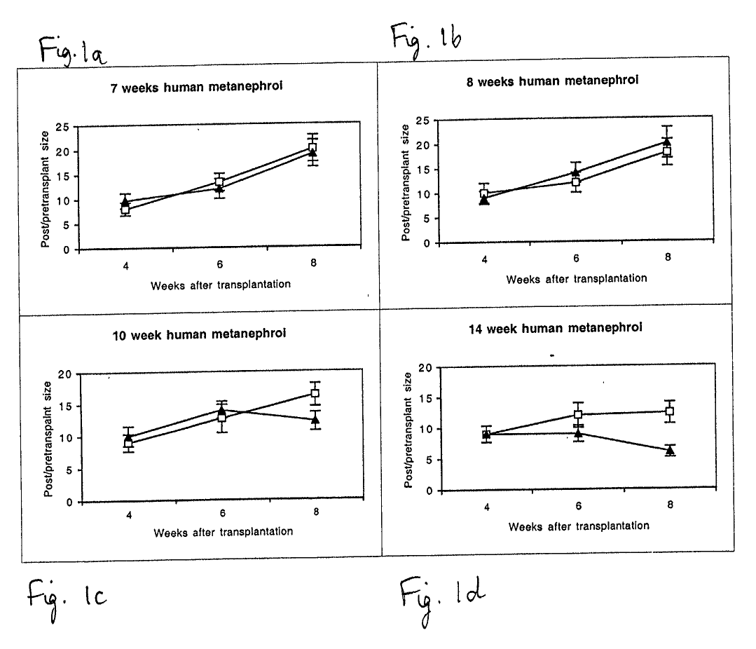

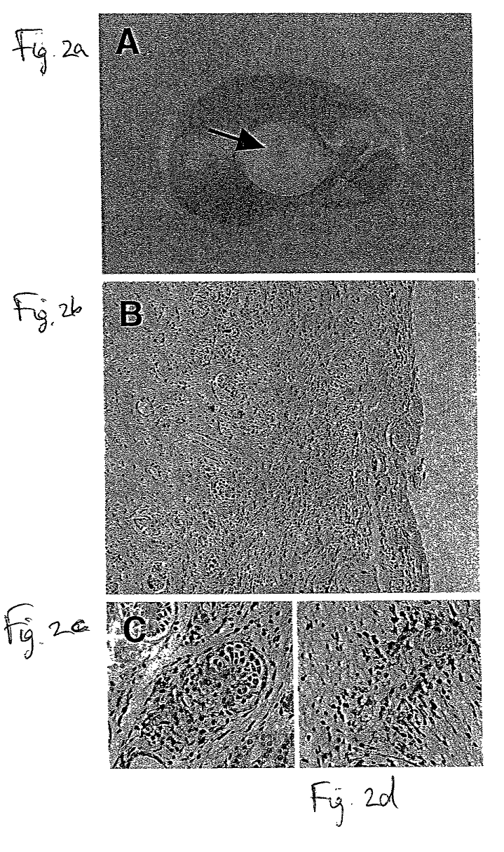

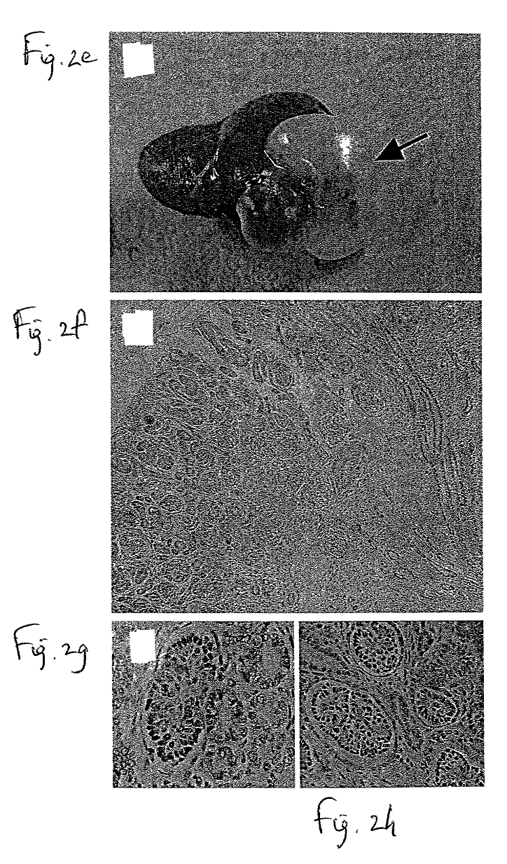

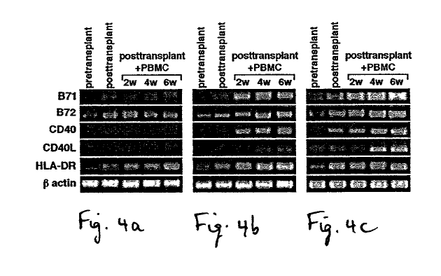

In contrast, under identical conditions, transplants of nephric tissue from 14-week fetuses were not rejected at 4 weeks posttransplant but rather significant growth of all transplants was observed (FIG. 1d) despite these being infiltrated with an average of 39.8±7.8 human T lymphocytes per microscopic HPF (Table 1). Nevertheless, analysis of such transplants at 6 and 8 weeks posttransplant revealed graft deterioration (Table 1, FIGS. 2a-d). Cellular infiltration and overall tissue deterioration are depicted in FIG. 2b and FIGS. 2c-d depict representative destruction of tubules and glomeruli, respectively. Transplant growth was also shown to be halted 8 weeks posttransplant, as demonstrated by average transplant size ratios of 6.2±0.9 versus 12.3±1.8 (p<0.01) for transplants from PBMC-infused and non-PBMC-infused hosts, respectively (Table 1, FIG. 1d). These and similar findings obtained from analysis of grafts of nephric tissue from 10-week fetuses (Table 3, FIG. 4c) showed that allogeneic human PBMC induced delayed rejection of grafts at these stages of nephrogenesis compared to grafts of adult-stage kidney tissue.

TABLE 1
In vivo interaction of human developing nephric tissue transplants
with allogeneic human PBMC

| Age of | No. | Infiltration¹ | | | |
|---|---|---|---|---|---|
| donor | of | (no. of CD3⁺ | Differentiation² | | Graft rejection³ |
| (wk) | mice | cells) | Glomeruli | Tubuli | (growth ratio) |
| 7 | 3 | 0 | 4.5 ± 0.5 | 18.0 ± 1.7 | 19.0 ± 2.7 |
| | | | | | no tissue |
| | | | | | damage |
| 8 | 3 | 0 | 4.7 ± 0.7 | 17.5 ± 2.3 | 20.3 ± 3.1 |
| | | | | | no tissue |
| | | | | | damage |
| 10 | 5 | 17.5 ± 3.5 | 6.2 ± 0.9 | 23.3 ± 2.4 | 12.2 ± 1.4 |
| (delayed | | | | | minimal tissue |
| rejection) | | | | | damage |
| 14 | 5 | 39.8 ± 7.8 | 2.1 ± 0.3 | 9.0 ± 0.4 | 6.2 ± 0.9 |
| (delayed | | | | | tissue damage |
| rejection) | | | | | |
| Adult⁴ | 5 | 87.2 ± 13.5 | — | — | Rapid graft |
| | | | | | destruction and |
| | | | | | rejection |

¹Data was obtained by immunostaining with anti-human CD3 at 4 weeks posttransplant and counting of CD3⁺ cells in 10 consecutive HPF (100× magnification) per transplant in 3 transplants per group.
²Differentiation was evaluated 6-8 weeks posttransplant by counting the number of mature glomeruli and tubuli in 10 consecutive HPF (40× magnification) per transplant in 3 transplants/group. No glomeruli and tubuli exist in pretransplant tissue of embryos of less than 8 weeks of gestation.
³Both histology (H + E) and transplant growth were evaluated. Transplant growth (formulated as posttransplant:pretransplant diameter ratio) was compared to that of control transplants subjected to PBMC infusion. At least 3 transplants were assessed in each group. At 8 weeks posttransplant growth was significantly reduced in transplants originating from 10- and 14-week fetuses compared to respective controls (see FIG. 1 and text).
⁴All adult kidney transplants were massively infiltrated and severely damaged at 4 weeks posttransplant, indicating acute cellular rejection as previously demonstrated (Dekel B. etal. (1997) Transplantation 64:1541; Dekel B. etal. (1999) Int Immunol. 11:1673).
Data are given as mean ± SEM

Transplants of nephric tissue from 7-week embryos or 8-week fetuses, on the other hand, displayed sustained growth rate profiles identical to those of transplants from non-PBMC infused mice (Table 1, FIGS. 1a and 1b, respectively). Differentiation of nephric tissue from 8-week fetuses into functional mature nephric organs, free of any signs of rejection by adoptively transferred human immune effector cells, was also clearly evident (Table 1, FIGS. 2e-h). FIG. 2e depicts the massive growth of such a graft into a nephric organ displaying a typical kidney morphology. Microscopic analysis of the grafts clearly demonstrated differentiation of well-formed glomeruli and tubuli in the absence of lymphocytic infiltrate (FIGS. 2f-h).

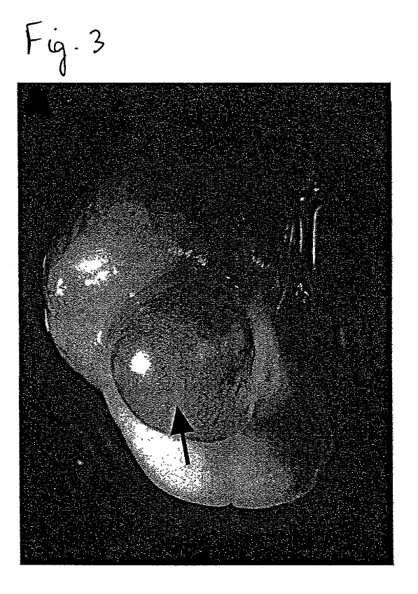

Nephric tissue grafts derived from 8-week fetuses were found to form large fluid-filled cysts (FIG. 3) posttransplant containing significantly higher levels of the renal metabolic byproducts urea nitrogen and creatinine than the serum of the transplanted mice (Table 2).

TABLE 2
Urea nitrogen and creatinine levels in cyst fluid
produced by human nephric tissue transplants and in serum and
bladder urine of transplanted mice.

| | Serum | Cyst fluid | Bladder urine |
|---|---|---|---|
| Urea N (mg/dl) | 37.0, 57.0 | 366, 665 | 4445, 4146 |
| Creatinine (mg/dl) | 0.4, 0.5 | 8.3, 6.3 | 48.8, 51.2 |

Results from two transplants are shown

Such production of urine-like fluid clearly showed that the grafts had developed into nephric organs having renal function. The lower urinary marker concentrations measured in cyst fluid compared to those in bladder urine are due to the fact that the cyst fluid was produced by nephric tissue at an immature developmental stage during which renal function is only partially developed whereas bladder urine is a product of fully functional adult-stage kidneys.

As shown in FIG. 4, PCR analysis did not detect expression of CD40 or CD40L mRNA in transplants of nephric tissue from 8-week human fetuses for up to 6 weeks posttransplant (FIG. 4a). In contrast such expression was detected in transplants of nephric tissue from 14- and 22-week fetuses by 4 weeks posttransplant (FIGS. 4b and 4c, respectively). In addition, B7-1 expression following transplantation and PBMC infusion was found to be significantly down-regulated in transplants of nephric tissue from 8-week fetuses (FIG. 4a) compared to transplants of nephric tissue from 14- and 22-week fetuses (FIGS. 4b and 4c, respectively).

This pattern of co-stimulatory molecule gene expression is therefore consistent with the in vivo data demonstrating complete absence of immune responses by human allogeneic effectors against transplants of human nephric tissue from 7- or 8-week human fetuses and thereby provides the mechanism underlying the functionality of the present invention.

In summary, these results therefore clearly demonstrate that transplanted grafts of nephric tissue from 7- to 8-week human embryos or fetuses have the capacity to differentiate into fully formed, vascularized and functional nephric organs whose growth and development is fully tolerated by allogeneic human PBMC. The finding that such early gestational nephric tissues represent the optimal stage for transplantation highlights the fact that results from prior art experiments could not be applied to nephric tissue transplantation. These prior art studies, involving transplantation of developing pancreatic tissues suggested, in sharp contrast, that end-gestational developing tissues were found to be optimal for transplantation (Otonkoski T. et al.(1999) Transplantation 68(11):1674).

Thus, this aspect of the method of the present invention represents a dramatic improvement over prior art methods of utilizing human nephric allograft transplantation to treat human kidney disease since prior art animal studies either did not demonstrate graft tolerance in the presence of human immune effects or prior art applied approaches required life-time administration of highly toxic immunosuppressant agents to prevent allograft rejection.

### Example 2

### Nephric Tissue Transplants from 4 Week-Old Porcine Fetuses Develop into Morphologically Differentiated, Functional Nephric Organs Which are Fully Tolerated by Xenogeneic Human PBMC

Treatment of kidney disease via transplantation of human kidneys is limited by the availability of matching donor organs. One promising solution to this obstacle is to utilize xenogeneic nephric grafts, such as porcine metanephric grafts, which are considered to be an optimally compatible alternative to human grafts for transplantation due to these avoiding hyperacute rejection as a virtue of their being vascularized by host vessels instead of donor vessels, as would be the case when transplanting solid organ grafts (D. P. Hyink et al. (1996) Am J Physiol. 270:F886; B. Robert et al. (1996) Am J Physiol. 271:F744). Thus, minimization, or preferably complete avoidance, of porcine nephric graft rejection by human immune cells constitutes a highly desired therapeutic goal for treatment of kidney disorders. Prior art approaches have shown that developing nephric tissue grafts induce attenuated alloimmune responses in comparison to adult-stage kidney allografts. While conceiving the present invention, the inventors hypothesized that the earliest developmental stage during which developing nephric tissues are sufficiently differentiated to develop into functional nephric organs following transplantation corresponds to the developmental stage during which rejection of such grafts can be optimally minimized or, ideally, completely avoided.

Therefore, identification of such an optimal stage of development in porcine nephric tissue was determined by transplantation thereof, at various stages of development, into immunodeficient mice reconstituted with xenoreactive human PBMC, as described below.

Materials and Methods:

Preparation of murine transplant hosts: Performed as per Example 1.

Harvesting of nephric tissue: Nephric tissue from 3 to 8 week-old porcine fetuses and from adult porcine kidney tissue, were obtained with the assistance of the Lahav Institute for Animal Research, Kibbutz Lahav, Israel. Porcine metanephroi were surgically dissected from fetuses using previously described techniques (Rogers S, et al. (1998) Kidney Int. 54:27).

Transplantation of nephric tissue: Performed as per Example 1.

Infusion of engrafted mice with human PBMC: Performed as per Example 1.

Analysis of graft infiltration, growth and differentiation: Performed as per Example 1.

Analysis of graft renal function: Analysis of graft renal function was performed via detection and quantitation of the renal function markers urea nitrogen and creatinine in fluid collected from large cysts formed by intra-abdominal grafts of nephric tissue from 4-week embryos transplanted in conjunction with infusion of xenoreactive human PBMC. Levels of renal function markers were measured in cyst fluid at 8 weeks posttransplant and were compared to those measured in the serum and bladder urine of transplanted mice.

Experimental Results:

During preliminary experiments to establish baseline experimental conditions, infusion of 10⁸ human mononuclear cells was determined to be the minimal number capable of inducing complete rejection of porcine adult nephric tissue transplants engrafted into recipient mice (data not shown). Four weeks following such infusion, transplants were found to be massively infiltrated and graft tissue destruction and rejection were apparent (Table 3).

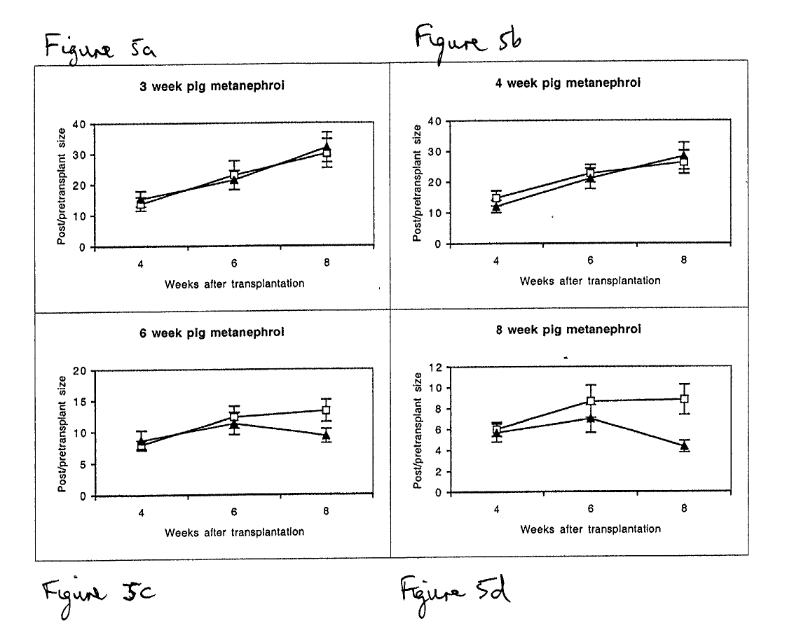

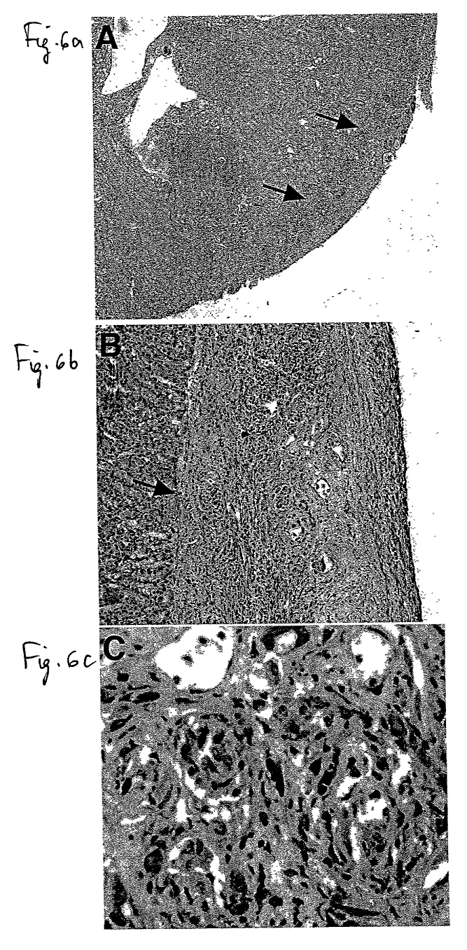

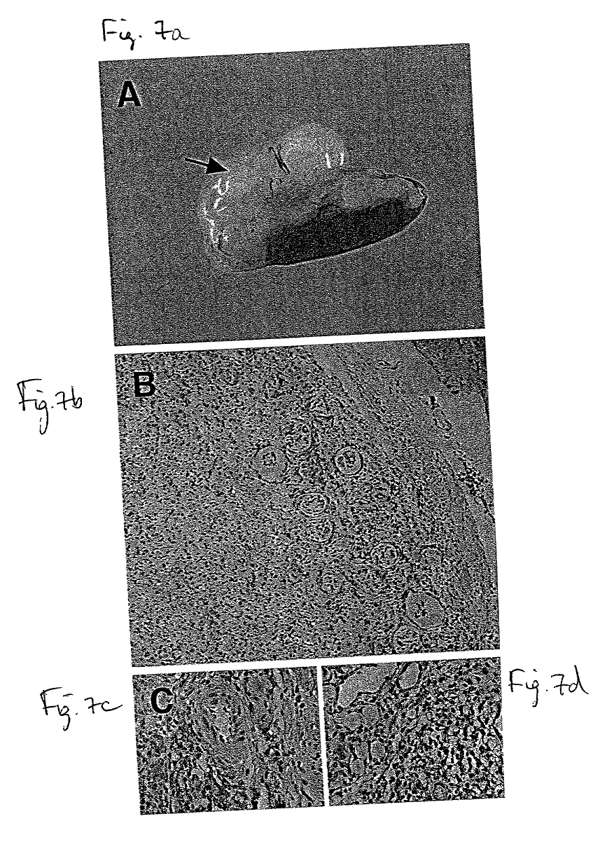

In contrast, under identical conditions, transplants of nephric tissue from 8-week fetuses displayed a sustained growth profile, identical to that of grafts from non-PBMC infused mice (FIG. 5), instead of being rejected, despite being infiltrated with an average of 40.5±6.7 human T lymphocytes per microscopic HPF (Table 3) and despite displaying destruction of nephric parenchyme tissue by human T cells (FIG. 6c).

TABLE 3
In vivo interaction of porcine nephric tissue transplants with
human PBMC

| | | | | | Graft rejection³ |
|---|---|---|---|---|---|
| | | | | | (post-:pre- |
| Age of | No. | Infiltration² | | | transplant |
| donor | of | (relative no. | Differentiation² | | diameter |
| (wk) | mice | CD3⁺ cells) | Glomeruli | Tubuli | growth ratio) |
| 3 | 9 | None | 5.9 ± 1.0 | 29.7 ± 3.8 | 31.7 ± 4.9 |
| | | | | | no tissue |
| | | | | | damage |
| 4 | 9 | None | 7.0 ± 1.0 | 35.5 ± 5.1 | 28.3 ± 4.4, |
| | | | | | no tissue |
| | | | | | damage |
| 6 | 6 | 22.7 ± 2.9 | 5.0 ± 0.6 | 21.6 ± 3.9 | 9.3 ± 1.1, |
| | | | | | minimal |
| | | | | | tissue damage |
| 8 | 6 | 40.5 ± 6.75 | 2.8 ± 0.5 | 11.9 ± 2.6 | 4.3 ± 0.6, |
| (delayed | | | | | tissue damage |
| rejection) | | | | | |
| Adult⁴ | 6 | 61.3 ± 8.3 | — | — | Graft |
| (delayed | | | | | destruction |
| rejection) | | | | | and rejection |

¹Data was obtained by immunostaining with anti-human CD3 at 4 weeks posttransplant and counting of CD3⁺ cells in 10 consecutive HPF (100× magnification) per transplant in 3 transplants per group.
²Differentiation was evaluated 6-8 weeks posttransplant by counting the number of mature glomeruli and tubuli in 10 consecutive HPF (40× magnification) per transplant in 3 transplants per group.
³Both histology (H + E) and transplant growth were evaluated. Transplant growth (formulated as posttransplant:pretransplant diameter ratio) was compared to that of non-PBMC-infused control transplants. At least 3 transplants were assessed in each group. At 8 weeks posttransplant growth was significantly reduced in transplants of nephric tissue from 6-week embryos and 8-week fetuses compared to respective non-PBMC-infused controls (see FIG. 5 and text).
⁴All adult nephric tissue transplants were massively infiltrated and severely damaged at 4 weeks posttransplant.
Data are given as mean ± SEM

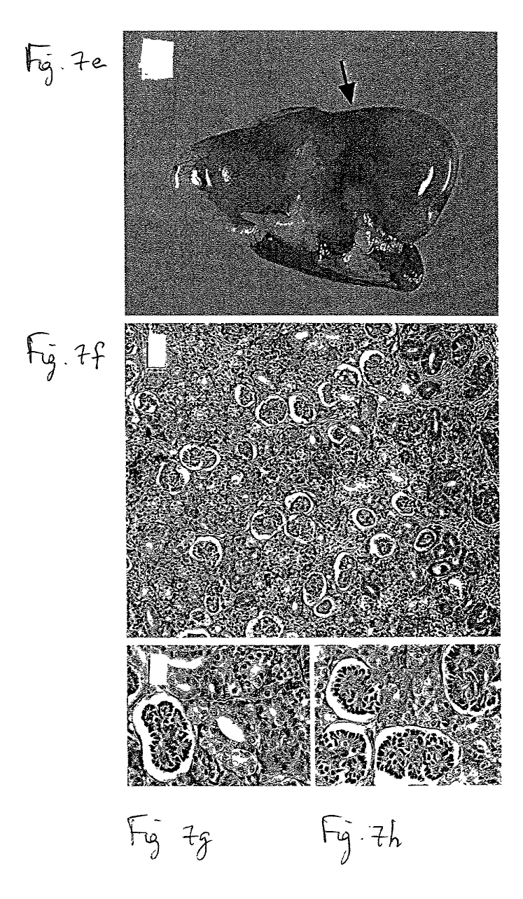

Analysis at 6 and 8 weeks posttransplant nevertheless indicated that 5 of 6 transplants displayed signs of rejection (Table 3 and FIGS. 7a-d). FIG. 7b depicts destruction of glomeruli and tubuli and general graft deterioration. FIGS. 7c and 7d depict destruction of blood vessels and tubules by human T cells, respectively. Transplant growth was also shown to be halted 8 weeks posttransplant, as demonstrated by average transplant size ratios of 4.3±0.6 versus 7.3±1.1 (p<0.02) for transplants from PBMC-infused or non-PBMC-infused hosts, respectively (Table 3, FIG. 5d). These and similar findings obtained from analysis of grafts of nephric tissue from 6-week embryos (Table 3, FIG. 5c) showed that xenoreactive human PBMC-induced rejection of grafts at these stages of nephrogenesis was clearly delayed compared to that of adult-stage nephric tissue grafts.

Transplants of nephric tissue from 3 and 4-week porcine embryos, on the other hand, displayed sustained growth rate profiles identical to those of transplants from non-PBMC reconstituted mice (Table 3 and FIGS. 5a and 5b, respectively). Differentiation of nephric tissue from 4-week embryos into mature nephric organs, free of any signs of rejection by xenoreactive human immune effectors, was also clearly evident (Table 3, FIGS. 7e-h). FIG. 7e depicts the massive growth of such a graft into a nephric organ displaying a typical kidney morphology and external vascular beds. Microscopic analysis of the grafts clearly demonstrated differentiation of well-formed glomeruli and tubuli (7.0±1.0 glomeruli and 35.5±5.1 tubuli per HPF) in the complete absence of any lymphocytic infiltrate or signs of tissue rejection (FIGS. 7f-h).

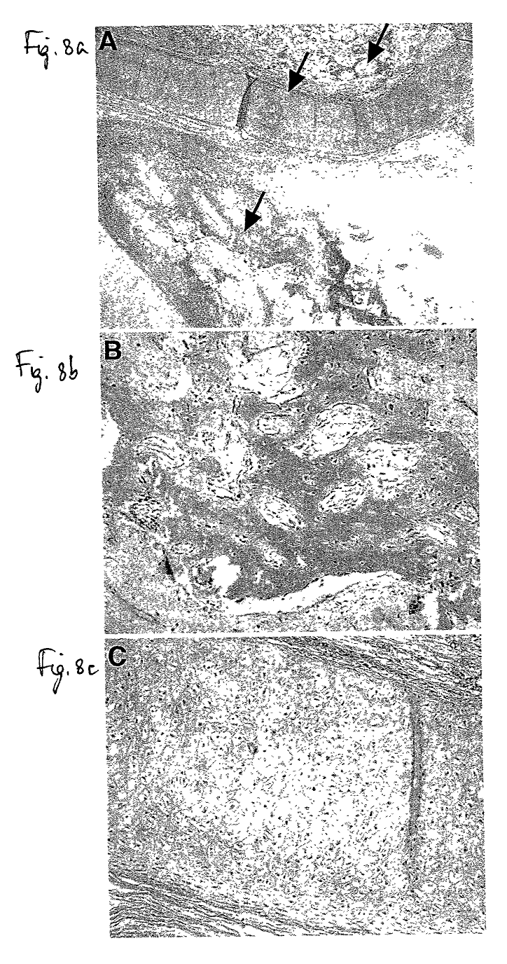

Analysis of transplants of nephric tissue from 3-week embryos indicated that 5 of 9 grafts had developed into large teratomas that, while containing a few glomeruli and tubuli, contained differentiated derivatives, such as cartilage and bone (FIG. 8). In contrast, none of the 4-week embryo nephric tissue transplants developed into teratomas.

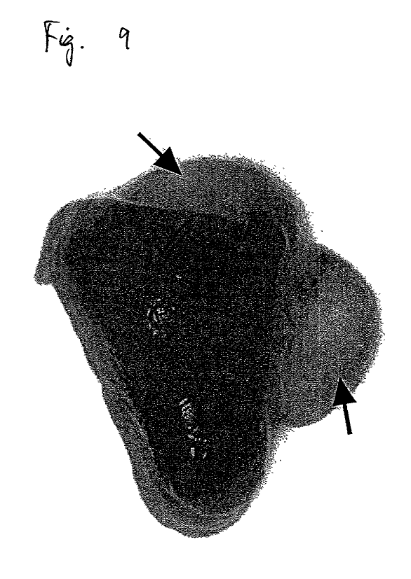

Four-week embryo nephric tissue grafts were found to form large fluid-filled cysts (FIG. 9) containing significantly higher levels of the renal metabolic byproducts urea nitrogen and creatinine than the serum of the transplanted mice (Table 4). Such production of urine-like fluid clearly showed that the grafts had developed into nephric organs having renal function. The lower urinary marker concentrations measured in cyst fluid compared to those in bladder urine are due to the fact that the cyst fluid was produced by nephric tissue at an immature developmental stage during which renal function is only partially developed whereas bladder urine is a product of fully functional adult-stage kidneys.

TABLE 4
Urea nitrogen and creatinine levels in cyst fluid
produced by porcine nephric tissue transplants and in serum and
bladder urine of transplanted mice.

| | Serum | Cyst fluid¹ | Bladder urine² |
|---|---|---|---|
| Urea N (mg/dl) | 43.7 ± 6.2 | 519 ± 182 | 4268 ± 464 |
| Creatinine (mg/dl) | 0.47 ± 0.04 | 7.2 ± 2.5 | 56.7 ± 8 |

¹p < 0.001 for measurement of urea N and creatinine in serum
²p < 0.001 for measurement of urea N and creatinine in cyst fluid

These experiments therefore succeeded in identifying nephric tissue from 4 week-old porcine embryos as representing the optimal stage of development for transplants capable of developing into fully formed, vascularized and functional nephric organs whose growth and development is completely tolerated and unaffected by xenoreactive human PBMC. Thus, the porcine nephric tissue grafts of the present invention can be employed to efficiently treat kidney disease without any form of immunosuppression whatsoever. As such, the method of the present invention constitutes a dramatic improvement over prior art methods of treating kidney disease using recipient-non-syngeneic porcine nephric tissue grafts which mandatorily require immunosuppressive treatment with highly toxic agents producing undesirable side-effects.

### Example 3

### Minimal Immunosuppression Enables Transplants of Nephric Tissue from 7- to 8-Week Human Fetuses or 4-Week Porcine Embryos to Treat Human Kidney Disease

While reducing the present invention to practice, as shown in Examples 1 and 2 respectively, transplants of nephric tissue from 7- to 8-week human fetuses or 4-week porcine embryos develop into morphologically differentiated, functional nephric organs which are fully tolerated by allo- or xeno-reactive human immune effectors, respectively, in the absence of any form of adjunct immunosuppressive treatment whatsoever.

Thus, transplants of the human and porcine nephric tissues mentioned hereinabove, being at a developmental stage during which tolerance thereof by allo- or xeno-reactive human immune effectors, respectively, is maximal, are utilized to treat human kidney disease with minimal adjunct immunosuppressive treatment in cases where such treatment is preferred.

As such, this aspect of the method of the present invention represents a great improvement over prior art methods of utilizing allogeneic human or xenogeneic porcine nephric tissue transplants to treat human kidney disease since it enables successful transplantation by administration of minimal levels of powerful immunosuppressant drugs, producing highly undesirable side-effects.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

What is claimed is:

1. A method of treating a kidney disease in a subject, the method comprising transplanting into the subject a graft of human nephric tissue being at a stage of differentiation corresponding to 4 to 10 weeks of gestation, thereby treating the kidney disease in the subject.

2. The method of claim 1, wherein said graft is selected not substantially displaying expression of CD40, CD40L, or both CD40 and CD40L.

3. The method of claim 2, wherein said selection is effected via RT-PCR analysis.

4. The method of claim 1, wherein said graft is selected displaying less expression of at least one molecule than nephric tissue of human 14 week-old fetuses.

5. The method of claim 4, wherein said selection is effected via RT-PCR analysis.

6. The method of claim 4, wherein said at least one molecule is capable of stimulating or enhancing an immune response.

7. The method of claim 4, wherein said at least one molecule is a lymphocyte coreceptor or a lymphocyte coreceptor ligand.

8. The method of claim 4, wherein said at least one molecule is B7-1, CD40 or CD40L.

9. The method of claim 1, wherein the subject is a human.

10. The method of claim 1, wherein the graft of human nephric tissue is transplanted into the renal capsule, the kidney, the testicular fat, the sub-cutis, the omentum or the intra-abdominal space of the subject.

11. The method of claim 1, further comprising treating the subject with an immunosuppressive regimen, thereby promoting engraftment of the graft of human nephric tissue in the subject.

12. The method of claim 11, wherein said treating the subject with an immunosuppressive regimen is effected prior to, concomitantly with or following said transplanting into the subject the graft of human nephric tissue.

13. The method of claim 11, wherein said treating the subject with an immunosuppressive regimen is effected by administration of an immunosuppressant drug and/or administration of an immune tolerance-inducing cell population.

14. The method of claim 1, wherein said stage of differentiation corresponds to 5 to 9 weeks of gestation.

15. The method of claim 1, wherein said stage of differentiation corresponds to 6 to 9 weeks of gestation.

16. The method of claim 1, wherein said stage of differentiation corresponds to 7 to 8 weeks of gestation.

17. The method of claim 1, wherein said stage of differentiation corresponds to 7 weeks of gestation.

18. The method of claim 1, wherein said stage of differentiation corresponds to 8 weeks of gestation.

19. A method of treating a kidney disease in a subject, the method comprising transplanting into the subject a graft of porcine nephric tissue being at a stage of differentiation corresponding to 3 to 6 weeks of gestation, thereby treating the kidney disease in the subject.

20. The method of claim 19, wherein said, graft is selected not substantially displaying expression of CD40, CD40L or both CD40 and CD40L.

21. The method of claim 20, wherein said selection is effected via RT-PCR analysis.

22. The method of claim 19, wherein said graft is selected displaying less expression of at least one molecule than nephric tissue of porcine fetuses at a developmental stage equivalent to that of nephric tissue of human 14 week-old fetuses.

23. The method of claim 22, wherein said selection is effected via RT-PCR analysis.

24. The method of claim 22, wherein said at least one molecule is capable of stimulating or enhancing an immune response.

25. The method of claim 22, wherein said at least one molecule is a lymphocyte coreceptor or a ligand of a lymphocyte coreceptor.

26. The method of claim 22, wherein said at least one molecule is B7-1, CD40 or CD40L.

27. The method of claim 19, wherein the subject is a human.

28. The method of claim 19, wherein the graft of porcine nephric tissue is transplanted into the renal capsule, the kidney, the testicular fat, the sub-cutis, the omentum or the intra-abdominal space of the subject.

29. The method of claim 19, further comprising treating the subject with an immunosuppressive regimen, thereby promoting engraftment of the graft of porcine nephric tissue in the subject.

30. The method of claim 29, wherein said treating the subject with an immunosuppressive regimen is effected prior to, concomitantly with or following said transplanting into the subject the graft of porcine nephric tissue.

31. The method of claim 29, wherein said treating the subject with an immunosuppressive regimen is effected by administration of an immunosuppressant drug and/or administration of an immune tolerance-inducing cell population.

32. The method of claim 19, wherein said stage of differentiation corresponds to 4 to 5 weeks of gestation.

33. The method of claim 19, wherein said stage of differentiation corresponds to 4 weeks of gestation.

34. The method of claim 19, wherein said stage of differentiation corresponds to 5 weeks of gestation.

35. A method of evaluating the transplantation suitability of a tissue explant or cell culture comprising testing cells of the tissue explant or cells of the cell culture for expression of at least one molecule, thereby evaluating the transplantation suitability of the tissue explant or cell culture.

36. The method of claim 35, wherein said testing is effected via RT-PCR analysis.

37. The method of claim 35, wherein said at least one molecule is capable of stimulating or enhancing an immune response.

38. The method of claim 35, wherein said at least one molecule is a lymphocyte coreceptor or a ligand of a lymphocyte coreceptor.

39. The method of claim 35, wherein said at least one molecule is CD40, CD40L or B7-1.
